(19) United States
(12) Patent Application Publication
(10) Pub. No.: US 2007/0100360 A1
(43) Pub. Date: May 3, 2007

(54) Combined dental floss and tongue scraper

(76) Inventor: Susanna Lee, Taipei (TW)

Correspondence Address: LEONG C LEI, PMB # 1008, 1867 YGNACIO VALLEY ROAD, WALNUT CREEK, CA, 94598, US

(21) Appl. No.: 11/263,940

(22) Filed: Nov. 2, 2005

(51) Int. Cl. A61B 17/24

(52) U.S. Cl. 606/161

(57) ABSTRACT

A combined dental floss and tongue scraper which comprises: a body portion having an end formed with a notch; a dental floss mounted across said notch; a holding portion being a recess formed on said body portion; and a scraper being a raised edge extending outwardly from another end of said body portion; whereby said dental floss can be used for cleaning spaces between teeth and said scraper for cleaning papillae of a tongue, thereby rendering it easy to carry and keep oral hygiene and cleanliness.

### BACKGROUND OF THE INVENTION

(a) Field of the Invention

The present invention relates to a combined dental floss and tongue scraper, in particular a structure with flossing and tongue scraping functions, which renders it easy for the user to carry, while cleaning scraps from in between the teeth and scraping the tongue clean, thus meeting the purpose of oral hygiene and cleanliness.

(b) Description of the Prior Art

The coating on the tongue is a moss-like substance attached to the surface of the tongue, constituted of filiform papilla and food scrapes, bacteria and saliva gathered within. Covering most of the tongue surface, it has a white appearance due to the high content of dead tissues. The filiform papilla grows as the amount of dead tissues increase, but can sometimes diminish or even disappear.

The formation of the tongue coating is derived from the mucus membrane on the surface of the tongue, an ongoing process of metabolism, and it displays the non-stop process of the fission, growth, differentiation, migration and flaking of the cell.

The above process is a balance of dynamism, and it maintains the thin layer of tongue coating of a normal person. The tongue coating accumulates when the filiform papilla grows and attaches itself to the tongue, thereby affecting the self-cleaning function of the tongue. This can occur when the chewing motion of the mouth and the mechanical rubbing on the tongue coating lessens as a result of appetite loss, reduction in food intake, or limiting the diet to soft and liquid foods, or dehydration due to fever resulting in decreased saliva secretion.

Also, tension can cause an increase in the acidity of the mouth and digestive tracts, facilitating the growth of the rosary fungus. The use of antibiotics to suppress certain bacteria can enhance the growth of certain microorganism. The above are all related to the formation of tongue coating.

The tongue coating is one of the sources of bad breath and oral disease. There is a need to regularly clean the accumulating tongue coating. The tongue scraping products currently being sold in the market cannot be widely used because they are not easy to carry, nor convenient to use. There is a need for the development of a combined dental floss and tongue scraper.

Although the invention referenced in Taiwan Patent Publication No. 351172, entitled “Dental Floss with Tongue Scraper” contains the dental floss and the tongue scraper, the user must bend the tongue scraper structure by hand, making it hard to hold.

Based on the above, the tongue scraper structure currently in use is not considered an ideal one.

Therefore, the present invention was developed by this inventor based on years of practical experience in research and development in the related field, and underwent countless actual designs and experiments.

### SUMMARY OF THE INVENTION

This invention is related to a combined dental floss and tongue scraper.

The present invention intends to provide an easy-to-use combined dental floss and tongue scraper. Its structure includes: a main body with an opening on the side; a dental floss placed on the opening in the main body; a holder on the main body forming a recess; a scraper that protrudes from one side of the main body. The afore-mentioned constitute the structure of a combined dental floss and tongue scraper, for cleaning the scraps from in between the teeth and scraping the tongue, for the purpose of oral hygiene and cleaning.

The foregoing object and summary provide only a brief introduction to the present invention. To fully appreciate these and other objects of the present invention as well as the invention itself, all of which will become apparent to those skilled in the art, the following detailed description of the invention and the claims should be read in conjunction with the accompanying drawings. Throughout the specification and drawings identical reference numerals refer to identical or similar parts.

Many other advantages and features of the present invention will become manifest to those versed in the art upon making reference to the detailed description and the accompanying sheets of drawings in which a preferred structural embodiment incorporating the principles of the present invention is shown by way of illustrative example.

### BRIEF DESCRIPTION OF THE DRAWINGS

FIG. 1 is a perspective view of the present invention;

FIG. 2 is a sectional view taken along line A-A of FIG. 1;

FIG. 3 illustrates how to use the present invention to clean spaces between the teeth; and

FIG. 4 illustrates how to use the present invention to clean papillae of the tongue.

### DETAILED DESCRIPTION OF THE PREFERRED EMBODIMENT

The following descriptions are of exemplary embodiments only, and are not intended to limit the scope, applicability or configuration of the invention in any way. Rather, the following description provides a convenient illustration for implementing exemplary embodiments of the invention. Various changes to the described embodiments may be made in the function and arrangement of the elements described without departing from the scope of the invention as set forth in the appended claims.

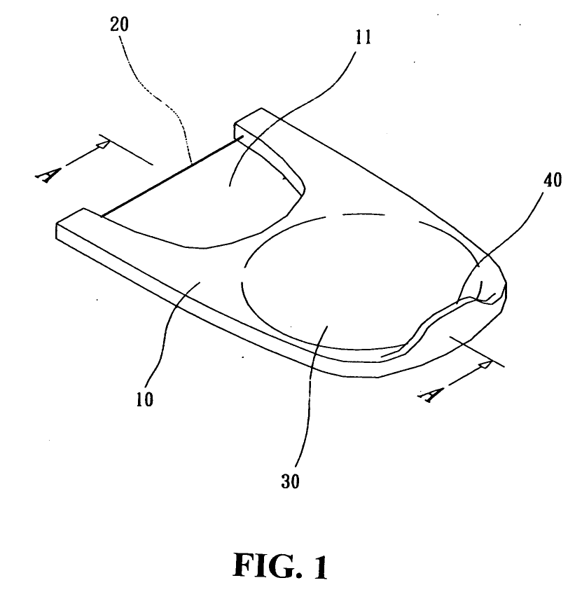

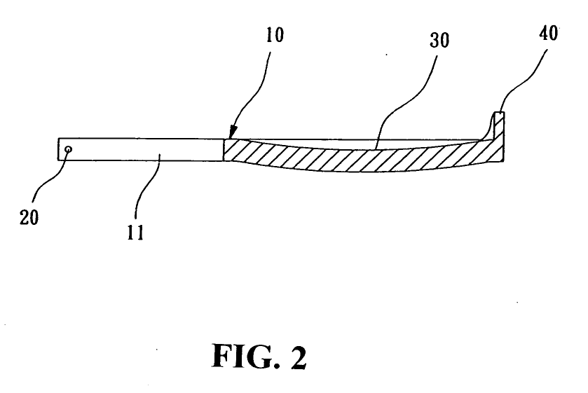

Referring to FIGS. 1 and 2, the combined dental floss and tongue scraper according to the present invention comprises a body portion 10, a dental floss 20 mounted at one end of the body portion 10, a holding portion 30, and a scraper 40 at the other end of the body portion 10. The user can use the dental floss 20 to clean the spaces between the teeth, and the scraper 40 to clean the papillae of the tongue thereby enabling the user to keep her or his mouth clean.

The body portion 10 has an end formed with a notch 11 across which is mounted a dental floss 20. The holding portion 30 is a shallow recess ergonomically configured to enable a user to hold conveniently, comfortably and firmly with her or his fingers. The scraper 40 is a raised edge extended upwardly from the other end of the body portion 10 and also ergonomically configured to enable a user to remove the papillae of her or his tongue.

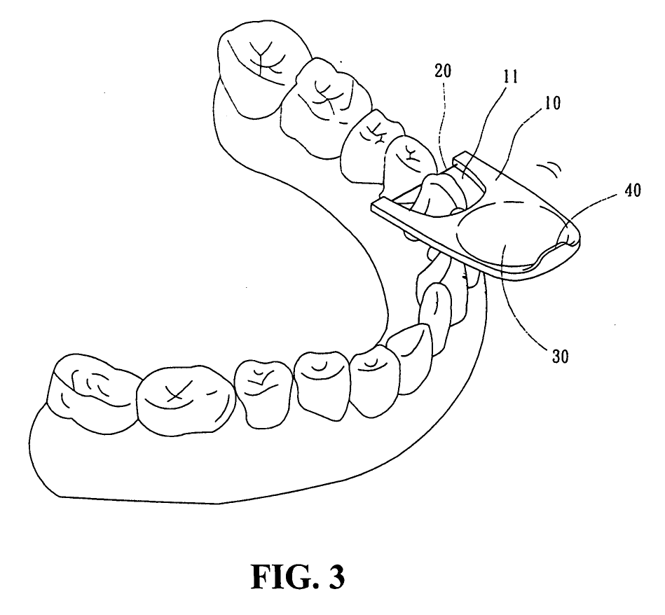

FIG. 3 illustrates how the user uses the dental floss to clean the spaces between her or his teeth.

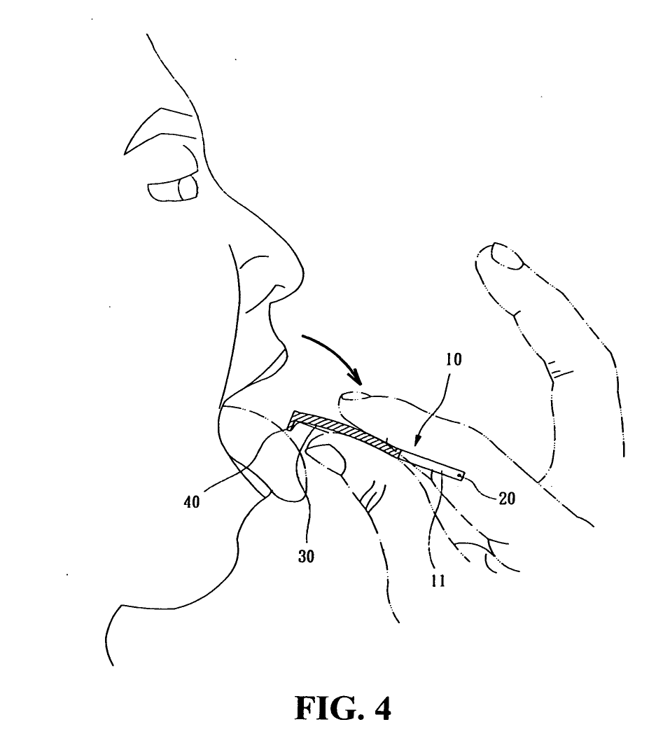

As can be seen in FIG. 4, when a user wants to remove the papillae of her or his tongue, simply hold the holding portion 30 with her or his fingers and turn the body portion 1 up side down to make the scraper 40 against the tongue and then scrape the tongue to remove the papilla.

In conclusion, the present invention can be used as a dental floss and a tongue scraper, so that it is unnecessary for one to carry two separate items thereby making it very convenient to carry and use.

It will be understood that each of the elements described above, or two or more together may also find a useful application in other types of methods differing from the type described above.

While certain novel features of this invention have been shown and described and are pointed out in the annexed claim, it is not intended to be limited to the details above, since it will be understood that various omissions, modifications, substitutions and changes in the forms and details of the device illustrated and in its operation can be made by those skilled in the art without departing in any way from the spirit of the present invention.

I claim:

1. A combined dental floss and tongue scraper comprising:
a body portion having an end formed with a notch;
a dental floss mounted across said notch;
a holding portion being a recess formed on said body portion; and
a scraper being a raised edge extending outwardly from another end of said body portion;
whereby said dental floss can be used for cleaning spaces between teeth and said scraper for cleaning papillae of a tongue.

2. The combined dental floss and tongue scraper as claimed in claim 1, wherein said holding portion is ergonomically configured to facilitate holding with fingers.

3. The combined dental floss and tongue scraper as claimed in claim 1, wherein said scraper is ergonomically configured to adapt to shape of a tongue.
